(19) United States
(12) Patent Application Publication
(10) Pub. No.: US 2009/0012348 A1
(43) Pub. Date: Jan. 8, 2009

(54) Magnetotherapy

(76) Inventor: Doyle Dean Simpson, Phelan, CA (US)

Correspondence Address: Maria Erlinda C. Sarno, Esq., P.O. Box 1023, Artesia, CA, 90702, US

(21) Appl. No.: 11/825,361

(22) Filed: Jul. 7, 2007

(51) Int. Cl. A61N 2/08

(52) U.S. Cl. 600/15

(57) ABSTRACT

A magnetotherapy device comprising a pair of magnets having a strap in between. The position of the magnetic pair along the strap can be changed as well as the number of magnetic pairs on the strap. One of the pair of magnets is housed inside a holder while the other pair is left exposed outside of the holder. This design allows easy reversal of the magnets when a change of polarity to the affected area/s is desired. The pair of magnets have flat outside and inside surfaces for maximum contact with the affected area/s of the body. The proposed treatment using the magnetotheray device comprises contacting the affected area/s with one polarity followed by contacting the same affected area/s with another polarity, different from the polarity of the previous contact.

Magnets have been used to relieve pain based on the belief that nerve impulses associated with pain are carried to the brain and magnetic therapy or magnetotherapy can impede the flow of these nerve impulses. Consequently, the magnet or magnets are placed at these areas which is/are not necessarily over the affected site or sites. For example, a migraine is recommended to be treated by placing the magnet over the chin while a shoulder pain is recommended to be treated by placing a magnet on the front and behind the shoulder. It has also been proposed that pain is relieved because the human blood, being replete with ions and electrolytes which are charged molecules, particles and the like, experience a force, including an aligning force when moving through a magnetic field. This motion causes generation of heat which widen the blood vessels and thereby allow increased flow of blood through them and cause it to gently churn. Magnets have also been used for its demonstrated effects on a cellular level such as that shown in the healing of tissue and fractures and also in increasing circulation at the affected area. Several patents on magnetic therapy have been issued, each of them proposing a different way of applying the magnets or applying this/these at different parts of the body.

Gilson Woo (U.S. Pat. No. 7,097,610B2) recommends applying the north pole of a magnet to a portion of the trunk and leaves this magnet for a period of time sufficient to elicit holistic effect of alleviation or cure in accordance with the total flux applied to the afflicted area. Woo differs from the proposed treatment because Woo does not subsequently apply the south pole surface of a magnet to the same area after exposure to the north pole. Anthony Bove et al. (U.S. Pat. No. 6,652,446) applies a magnetic sheet of material with alternating magnetic polarities to the affected area/s. Most magnetotherapy devices sold in the market are like Bove's which have alternating magnetic polarities, that is, both north and south poles are contacting the affected areas simultaneously.

Many different holders for magnets have been developed such as the belt disclosed by Bakst (U.S. Pat. No. 5,707,333) that can be worn around the waist of a user with the wide portion of the belt comprising an array of magnetic sources arranged by a supporting web. Gilson Woo (U.S. Pat. No. 7,097,610B2) discloses a wrap around belt to securely hold the magnets sewn-in or encased in fabric lining or adjustable Velcro type fasteners. James Snyder (U.S. Pat. No. 6,926,661B2) describes a magnet orientation assembly in combination with a mattress or mattress liner where an array of magnets of oppositely disposed poles can be maintained in a predetermined, operative orientation even as the individual moves throughout a night sleep or other rest period by using a support structure that also allows removal of magnets for cleaning, repairing or replacing. Brand N. Griffin, et al. (U.S. Pat. No. 6,554,787B1) disclosed a headband for treatment of headaches having multiple pockets at the interior of the headband for housing one or more heating, cooling, magnetic and vibratory elements to provide heat, cold, vibration and magnetism or a combination thereof to the head.

In all the above designs, the magnet/s can not be easily moved to another location because each magnet is housed in a pocket or are held by support structures. Further, the number of magnets can not be readily altered beyond the number of holders. Also, the polarity of the magnet facing the body can not be easily reversed.

It is therefore an object of this invention to provide a simple holder for magnets used in magnetotherapy that allow movement of the magnet along a strip of material thereby allowing adjustment of position to cater to the location and position of the affected area.

It is also an object of this invention to provide a magnet holder that can add or eliminate magnets according to the intended usage.

It is a further object of this invention to provide a holder that will allow a user to easily reverse the polarity of the magnet during the course of treatment.

It is also a further object of this invention to propose a method of magnetotherapy different from those currently practiced.

### SUMMARY OF THE INVENTION

The invention relates to a magnetotherapy device for alleviating pain, healing tissues, rebuilding fractures and increasing circulation of blood. This device comprises a pair of disc shaped magnets with flat inside and outside surfaces, a top magnet and a bottom magnet attached to each other having a strap sandwiched in between, the pair of magnet movable along a length of the strap; a non-magnetic holder having a magnetic strip attaching to the bottom magnet, leaving a surface of the same polarity of the top magnet exposed, the pair of magnets removable from the holder for easily reversing and replacing the bottom magnet with the top magnet; and, a fastener means for wrapping around the strap to a part of a human body, thereby contacting the exposed surface of the magnet having the same polarity with the part of the body. A second holder having a magnetic strip attaching to the top magnet can also be provided to cover the exposed surface of the pair of magnets before storage. The non-magnetic holders can have a hollow or a solid interior under the magnetic strip. A second strap with a fastener pairing with the fastener means of the strap sandwiched between the top and bottom magnets can also be provided to lengthen the strap. Instead of just one pair, a plurality of magnetic pairs can be provided along the strap, however, it is important that the exposed surfaces of all magnetic pairs have one uniform polarity. The magnet in this device has a magnetic flux density of 2700-5000 gauss. The strap is made of fabric or leather having a varying length or varying number of fasteners along the length of the strap. In the assembled device, the strap and the top magnet are above the holder.

A method for using the magnetotherapy device for alleviating pain, healing tissues, rebuilding fractures and increasing circulation of blood, comprises the steps of:

a) contacting a flat outside exposed surface of the top magnet having one polarity to an affected area of the body for an interval of time;

b) wrapping the strap of the magnetotherapy device around the affected area of the body and securing the pair of magnets with a fastener placed along a length of the strap;

c) reversing the polarity of the outside exposed surface of the magnet by pulling the pair of magnet from the holder through the strap, reversing the position of the pair of magnets, placing the top magnet to the bottom and the bottom magnet to the top by flipping over the pair of magnet, and placing one of the pair of magnets back inside the holder at the reversed position, thereby exposing a polarity different from the polarity in step a);

d) allowing the exposed outside surface of the magnet in step c) to contact the same affected area of the body for an interval of time;

e) repeating steps a)-d) for a total contact time of one hour;

f) discontinuing contact with the magnet for approximately two hours; and,

g) resuming steps a)-f) for a total treatment time of approximately twelve hours. The treatment is resumed when pain recurs or when cellular repair and increased circulation are needed. The affected area is contacted with a magnet having a magnetic flux density of 2700-5000 gauss. The contact time of the magnets with the affected area/s of the body is at least 10-15 minutes. It has been found for most cases that positive results are obtained when the affected area of the body is first contacted with a negative (−) polarity followed by a positive (+) polarity.

Other embodiments of the present invention will become readily apparent to those skilled in the art from the following detailed description, wherein it shows and describes only certain embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWING

Aspects of the present invention are illustrated by way of example, and not by way of limitation, in the accompanying drawings, wherein:

FIG. 1 is a perspective view of the magnetotherapy device.

FIG. 2 is an exploded view of the components of the magnetotherapy device.

FIG. 3 is a side view of the magnetotherapy device having a second holder covering the exposed surface of the magnetotherapy device for storage.

FIG. 4 shows the application of the magnetotherapy device on the shoulder and wrist.

FIG. 5 shows the application of two instead of one magnetotherapy device on the shoulder of a user.

### DETAILED DESCRIPTION OF THE INVENTION

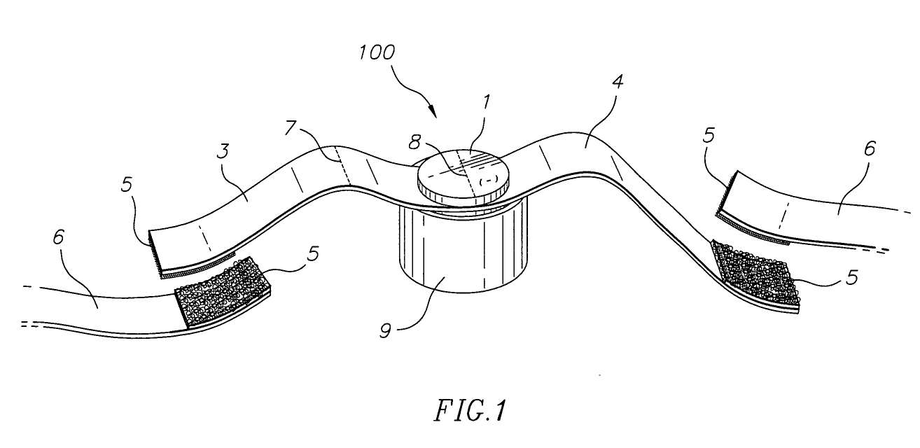

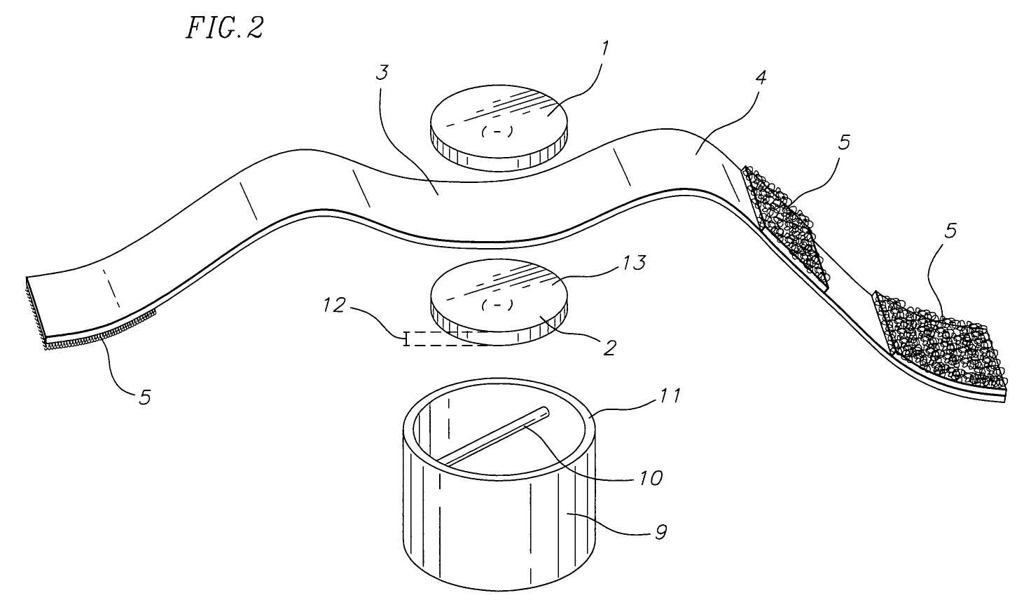

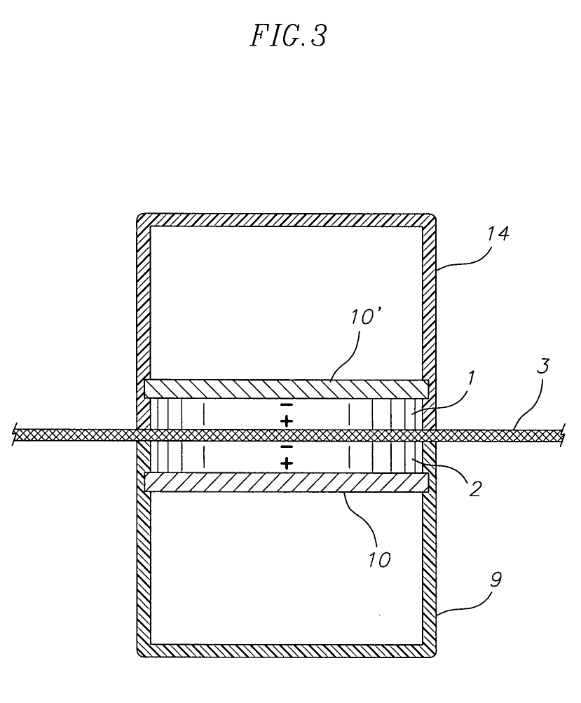

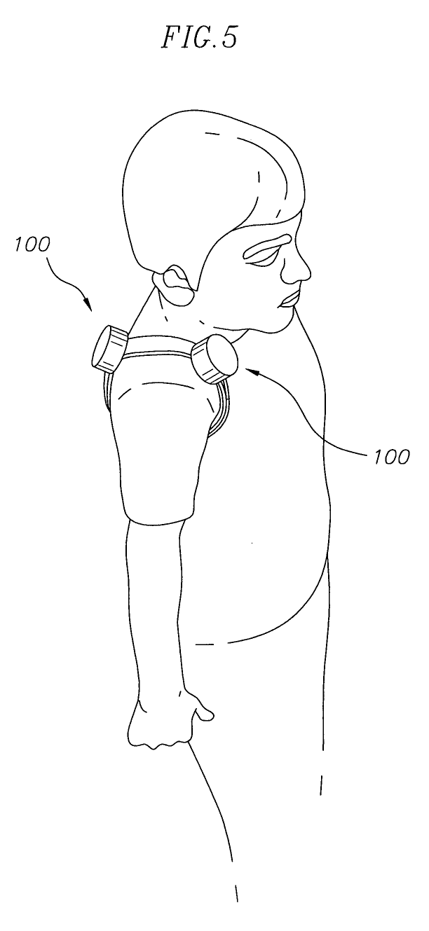

The magnetothery device 100 is shown in FIGS. 1 and 2. It comprises of two magnets 1 and 2, the magnets, sandwiching between them, a band or strap 3. The magnets, 1 and 2 are paired, herein also referred to as magnetic pair, and held in place at the chosen location along the strap 3 because of the intensity of the attraction between the two magnets, the positive pole (+), the north-seeking pole of magnet 1 attaching to the negative pole (−), the south-seeking pole of a magnet 2 despite the presence of a non-magnetic band or strap 3 in between the two magnets. In the provided drawings, magnet 1 is the top magnet while magnet 2 is the bottom magnet. In this application, it is recommended to have the negative pole of magnet 1 at the top exposed surface while the positive pole (+) of magnet 1 is placed at the interior side opposite the negative pole. A compass can be used to determine and ensure the polarity of the exposed surface of magnet 1. The compass needle points to the south pole of the magnet which has the negative polarity. The positive pole (+) of magnet 1 attaches to the negative pole (−) of magnet 2 as show in FIG. 3. Although the attraction between the two magnets are strong, their paired position can be transported along the length 4 of the strap 3 with a manual force greater than that exerted by the magnetic force. This design makes the positioning of the magnet flexible. Further, the strap 3 can have varying length for wrapping around different parts of the body or have several fasteners 5 along the length of the strap 3 to accomplish the same purpose as shown in FIG. 2. Although FIG. 2 shows only one extra Velcro connector at the strap, several of these can be placed depending upon the length of the strap. The length of the strap 3 can also be lengthened by joining a second strap 6 with a pairing fastener 5 such as Velcro, a hook and loop fastener, to the first strap 3 as shown in FIG. 1. The strap is recommended to have the same width 7 as the diameter/s 8 of the magnet/s. The magnets as shown here is disc type, with flat outside and inside surfaces. A flat surface is desired so it can have maximum surface to surface contact between the body and the magnet. Non-circular magnets with flat surfaces may also be used. Any type of magnets such as permanent magnets, for example rare earth magnet, magnet incorporating neodymium like NdFeB magnets, ceramic magnets and magnetic ferrites as well as electromagnet, radiomagnet and the like can be used so long as its magnetic flux density is between 2700 to 5000 gauss for optimal effect. The resulting magnetic flux is usually contributed by the size, shape and type of the magnetic material. There can also be more than one pair of magnets in the same strap as shown in FIG. 5 and up to a total magnetic flux density of approximately 10,000 gauss have been applied without observing any harmful effect.

To keep the positive (+) pole of the magnet 2 from being exposed and consequently also exerting a magnetic force on the body and its surroundings, a holder 9 made of non-magnetic material is used to house and cover magnet 2. The magnet 2 is kept in place inside the holder by a magnetic strip 10 running along the diameter of the holder 9 which attaches to magnet 2, at a position recessed from the top edge 11 of the holder as shown in FIGS. 1 and 2. A magnetic strip on the holder rather than a disc is preferred to minimize further conduction of magnetic force. The extent of the recess is comparable with the thickness 12 of the magnet 2. Consequently, after magnet 2 lodges at the recessed top portion inside the holder 9, the outside surface 13 is flushed with the top edge 11 of the holder. This set up keeps the strap 3 above the top edge 11 of the holder as shown in FIGS. 1 and 3. The body of holder 9 can have a hollow or solid interior under the magnetic strip 10 with a depth enough to minimize any magnetic force or attraction emanating from the outside surface of the holder 9 coming from magnet 2. A good holder will not attract outside magnetic particles at the outside bottom surface of the holder 9 opposite the end that is housing and holding magnet 2. For storing the magnetotherapy device 100, a second holder 14 which is a mirror image of holder 9 is placed on top of magnet 1 to cover the exposed surface of magnet 1 in a similar fashion as holder 9 covers the exposed surface of magnet 2 as shown in FIG. 3. The second holder also has a second magnetic strip 10′ to hold the holder 14 in place through the attraction between the magnetic strip 10′ and magnet 1. The second holder 14, like the first holder 9, also minimizes the magnetic force emanating to the outside surroundings from magnet 1. It is not critical whether magnet 1 or magnet 2 is inside holder 9 or holder 14. These can be interchanged since the treatment calls for reversing their positions as will be described.

The strap 3 is recommended to be made of a fabric material due to its softness and flexibility. An elastic fabric strap will also add to the adjustability of the device 100 in its ability to wrap around at different parts of the body in addition to the presence of fasteners like the hook and loop (Velcro) fastener, hook and eye, button, snaps and the like placed at different locations along the length 4 of the strap 3 as shown in FIG. 2. The strap can also be made of natural or synthetic leather fastening like a belt. To move a pair of magnet to another location along the length of the strap, a user grasps on both magnets 1 and 2 and pulls these towards the desired location while the other hand holding the strap, pulls the strap towards the opposite direction, until the desired position for the pair of magnets is reached.

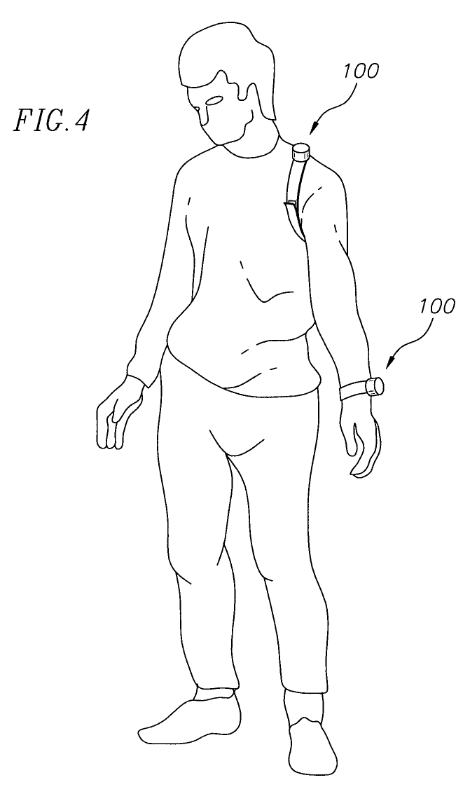

The construction of the magnetotherapy device 100 is tailored to the method of treatment proposed here which is different from the conventional method where only one pole of the same polarity or an array of magnets of multiple polarities are applied to the area of the body where alleviation of pain, cellular repair, increase circulation and/or bone fracture rebuilding and mending is or are desired. These areas of the body are collectively referred to herein as affected area. The claimed method exposes the affected area/s of the body to only one polarity at a time. The method of magnetotherapy comprises contacting a flat outside exposed surface of magnet 1 to the area of the body where alleviation of pain or cellular repair is desired for a period of at least 10-15 minutes. This duration can be longer and observance of strict time of application is not necessary because exposure to magnets are not known to be harmful to the body. The magnet has a north-south magnetic axes and is disposed substantially perpendicular to the surface of the body. The treatment starts with applying only one polarity to an affected area, herein usually the negative (−) pole for the duration of time indicated above. After at least 10-15 minutes, the magnets are reversed in position. The flat outside surface of magnet 2 instead of the magnet 1 is now exposed at the outside surface of the magnetotherapy device. As shown in FIG. 3, upon reversal, that is, after flipping over the pair of magnet, the positive (+) pole of magnet 2 is now the side exposed and this is contacted with the same affected area of the body where the negative (−) pole was previously applied for at least 10-15 minutes. This step would be difficult to undertake if the device is not constructed as described because it will be difficult to readily reverse the polarity with the existing magnet holders. Here, the user simply takes the adhered magnets 1 and 2 out of the holder 9 by using the strap 3 to easily pull them out of the holder and flipping the magnets such that magnet 2 is now on top of magnet 1 and the strap 3. Magnet 1 becomes the bottom magnet and is inserted into the recessed portion of the holder 9 with the positive (+) pole of magnet 1 flushed with the top edge 11 of holder 9. This process of alternating treatment with the negative pole followed by the positive pole is repeated until the total exposure time is approximately one hour. On some occasions, treatment with the positive (+) before the negative (−) polarity in the same alternating regimen also worked well. If more treatment is desired or needed, after one hour of exposure to alternating polarities, the user is then advised to take approximately a two hour break before applying the magnets again as described above. Either before or after a total of 12 hours of treatment, including here the 2 hours break time between applications of the magnet, the user should experience some alleviation of pain. The cellular effects, however, may not be detected readily without the use of some specialized instruments or tests. FIG. 4 shows the application of the magnets in the shoulder and wrist area. As stated above, there may be more than one magnetotherapy device used as shown in FIG. 5. When there are more than one magnetotherapy devices along the same strap, that is, more than one magnetic pairs, it is important that the exposed magnetic surfaces contacting the body are all of the same uniform polarity. The diameter of the magnetic discs may vary according the size of the area to be treated and the strength of the magnetic flux density desired. The pain may recur or cellular repair and increased blood circulation may be needed a few days, months or years after the first regimen of treatment. When this happens, the user resumes and repeats the treatment as described above. The number of times and duration of exposures depend upon the type and severity of the injury as well as the individual differences between one person to the other.

While the embodiments of the present invention have been described, it should be understood that various changes, adaptations, and modifications may be made therein without departing from the spirit of the invention and the scope of the claims.

I claim:

1. A magnetotherapy device for alleviating pain, healing tissues, rebuilding fractures and increasing circulation of blood, comprising:
a pair of disc shaped magnets, a top magnet and a bottom magnet attached to each other having a strap sandwiched in between, the pair of magnet movable along a length of the strap;
a non-magnetic holder having a magnetic strip attaching to the bottom magnet, leaving a surface of the top magnet of the same polarity exposed, the pair of magnets removable from the holder for easily reversing and replacing the bottom magnet with the top magnet; and,
a fastener means for wrapping around the strap to a part of a human body, thereby contacting the exposed surface of the magnet having the same polarity with the part of the body.

2. The magnetotherapy device of claim 1 further comprising a second holder having a magnetic strip attaching to the top magnet for covering the exposed surface of the pair of magnets and for storing the magnetotherapy device.

3. The magnetotherapy device of claim 1 wherein the non-magnetic holder has a hollow or a solid interior under the magnetic strip.

4. The magnetotherapy device of claim 1 further comprising a second strap with a fastener pairing with the fastener means of the strap sandwiched between the top and bottom magnets.

5. The magnetotherapy device of claim 1 further comprising a plurality of magnetic pairs along the strap, the exposed surfaces of all magnetic pairs having one uniform polarity.

6. The magnetotherapy device of claim 1 wherein the magnet has a magnetic flux density of 2700-5000 gauss.

7. The magnetotherapy device of claim 1 wherein the strap is made of fabric or leather having a varying length or varying number of fasteners along the length of the strap.

8. The magnetotherapy device of claim 1 wherein the magnets have flat inside and outside surfaces.

9. The magnetotherapy device of claim 1 wherein the strap and the top magnet are above the holder.

10. A method for alleviating pain, healing tissues and fractures and increasing circulation of blood using a magnetotherapy device having a pair of top and bottom magnet sandwiching a strap in between with a holder covering the bottom magnet, comprising the steps of:
a) contacting a flat outside exposed surface of the top magnet having one polarity to an affected area of the body for an interval of time;
b) reversing the polarity of the outside exposed surface of the magnet by reversing the position of the pair of magnets, placing the top magnet to the bottom inside the holder and the bottom magnet to the top, thereby exposing a polarity different from the polarity in step a);
c) allowing the exposed outside surface of the magnet in step b) to contact the same affected area of the body for an interval of time;
d) repeating steps a)-c) for a total contact time of one hour.

11. The method of claim 10 further comprising the steps of e) discontinuing contact with the magnet for approximately two hours; and, f) resuming steps a)-e) for a total treatment time of approximately twelve hours.

12. The method of claim 10 wherein the affected area is contacted with a magnet having a magnetic flux density of 2700-5000 gauss.

13. The method of claim 10 wherein the interval of treatment for steps a) and c) is at least 10-15 minutes.

14. The method of claim 10 wherein the affected area of the body is first contacted with a negative (−) polarity followed by a positive (+) polarity;

15. A method for alleviating pain, healing tissues, rebuilding fractures and increasing circulation of blood using a magnetotherapy device having a pair of top and bottom magnets sandwiching a strap in between with a holder covering the bottom magnet, comprising the steps of:
a) contacting a flat outside exposed surface of the top magnet having one polarity to an affected area of the body for an interval of time;
b) wrapping the strap of the magnetotherapy device around the affected area of the body and securing the pair of magnets with a fastener placed along a length of the strap;
c) reversing the polarity of the outside exposed surface of the magnet by pulling the pair of magnet from the holder through the strap, reversing the position of the pair of magnets, placing the top magnet to the bottom and the bottom magnet to the top by flipping over the pair of magnet, and placing one of the pair of magnets back inside the holder at the reversed position, thereby exposing a polarity different from the polarity in step a);
d) allowing the exposed outside surface of the magnet in step c) to contact the same affected area of the body for an interval of time;
e) repeating steps a)-d) for a total contact time of one hour;
f) discontinuing contact with the magnet for approximately two hours; and,
g) resuming steps a)-f) for a total treatment time of approximately twelve hours.

16. The method of claim 15 further comprising resuming the treatment when pain recurs or when cellular repair and increased circulation are needed.

17. The method of claim 15 wherein the affected area is contacted with a magnet having a magnetic flux density of 2700-5000 gauss.

18. The method of claim 15 wherein the interval of treatment for steps a) and d) is at least 10-15 minutes.

19. The method of claim 15 wherein the body is first contacted with a negative (−) polarity followed by a positive (+) polarity.
